(19) United States
(12) Patent Application Publication
(10) Pub. No.: US 2012/0302888 A1
(43) Pub. Date: Nov. 29, 2012

(54) ARRAY ULTRASOUND ENDOSCOPIC PROBE, A MANUFACTURE METHOD THEREOF AND A FIXING AND ROTATING DEVICE

(75) Inventors: Jiyan Dai, Hong Kong (CN); Kwok Fung Cheung, Hong Kong (CN); Dan Zhou, Hong Kong (CN); Yat Ching Chiu, Hong Kong (CN); Kwok Ho Lam, Hong Kong (CN); Yan Chen, Hong Kong (CN); Helen L. W. Chan, Hong Kong (CN)

(73) Assignee: The Hong Kong Polytechnic University, Hong Kong (CN)

(21) Appl. No.: 13/479,017

(22) Filed: May 23, 2012

(30) Foreign Application Priority Data
May 23, 2011 (CN) 201110132195.1

(51) Int. Cl. A61B 8/12; H01L 41/22

(52) U.S. Cl. 600/447; 29/25.35

(57) ABSTRACT

This invention relates to a radial array ultrasound endoscopic probe and its manufacture method and a fixing and rotating device. The probe comprises: a metal cylinder located at the center; a plurality of piezoelectric elements which are cut from piezoelectric ceramic or single crystal rings and arranged around the metal cylinder, said piezoelectric elements and said metal cylinder being provided with a backing material layer for absorbing sound therebetween, said piezoelectric elements being covered with a matching material layer on the outer side thereof, and said piezoelectric elements are filled with decoupling material therebetween; a plurality of coaxial cables correspondingly connected with the plurality of piezoelectric elements; and a circular lattice in a gear shape which sleeves over said metal cylinder and functions to arrange and separate the coaxial cables. In the invention, the plurality of piezoelectric elements are directly cut out from the piezoelectric ceramic or single crystal ring, thus non-concentricity of arrays, misalignment of arrays at interface, the probe being liable to damage, disengagement and break-off, etc., that may arise from forcedly curling multiple lays of materials of a specific thickness, are avoided.

### FIELD OF THE INVENTION

The present invention relates to the technical field of ultrasound endoscopy. In particular, it relates to a radial array ultrasound endoscopic probe, a manufacture method thereof and a fixing and rotating device.

### CROSS REFERENCE TO RELATED APPLICATIONS

This application claims benefit of Chinese patent application No. 201110132195.1, filed May 23, 2011, the disclosure of which is incorporated in its entirety by reference for all purposes.

### BACKGROUND OF THE INVENTION

In the technical field of ultrasound endoscopy, a radial scanning probe is widely used for image diagnosis for gallbladder, pancreas and alimentary canals. At early stage of its usage in 1980s, only 90-degree ultrasound imaging could be realized by means of ultrasound endoscopy. Later on, with the technical developments, 180-degree, and even 360-degree ultrasound imaging are realized.

The earliest 360-degree image is obtained through scanning by a single-element probe, which is mechanically driven by a motor to rotate inside the endoscope. Later on, ultrasonic images can be obtained by a radial array probe through 360-degree electronic scanning, in which a motor is dispensed with. Furthermore, imaging can be quickly conducted and thus-obtained images are clearer, which renders the radial array probe applicable in examining fine structures of inner organs and tissues. A radial array ultrasound endoscopic probe mainly includes a plurality of strip elements that are arranged parallel with the axial direction of the cylinder. The more the number of the elements is, the higher the resolution of resulting images.

At present, the technique for producing radial array ultrasound probes of small scale to be placed on the top end of the endoscopy is quite complex, therefore the probes of this type are seldom commercially available. Only a few manufacturers are capable of producing the probes of this type, thus price thereof is relatively high. In the prior manufacture method for probes, it is common to prepare planar elements first and then to curl them to a circular shape. The main disadvantage associated with this method lies in the fact that curling will probably lead to an imperfect cylindrical shape and at least one interface will certainly exist. Thus there may be aberrance in the resultant images. In addition, since layers in the element (such as the acoustic matching layer, the piezoelectric ceramic or single crystal, and the backing layer) are connected before curling, break-down and disengagement of different layers may occur due to stress arising from curling.

### BRIEF SUMMARY OF THE INVENTION

The technical problems to be solved by the invention is to propose a simple and reliable radial array ultrasound endoscopic probe, a manufacture method thereof and a fixing and rotating device which can overcome the drawbacks of the prior art that aberrance would occur due to breaking-off caused by that the radial array ultrasound endoscopic probe is made by manufacturing the plane array first and then curling the same.

In the first aspect of the invention, it is provided a radial array ultrasound endoscopic probe, comprising a metal cylinder located at the center; a plurality of piezoelectric elements which are cut from piezoelectric ceramic or single crystal rings and are arranged around the metal cylinder, said piezoelectric elements and said metal cylinder being provided with a backing material layer for absorbing sound therebetween, said piezoelectric elements being covered with a matching material layer on the outer side thereof, and said piezoelectric elements are filled with decoupling material therebetween; a plurality of coaxial cables correspondingly connected with the plurality of piezoelectric elements, the grounding wire of each coaxial cable being connected to the metal cylinder; and a circular lattice in a gear shape which sleeves over said metal cylinder and functions to arrange and separate the coaxial cables.

In the radial array ultrasound endoscopic probe according to the first aspect of the invention, the plurality of piezoelectric elements are cut from a piezoelectric ceramic ring or a piezoelectric single crystal ring.

In the radial array ultrasound endoscopic probe according to the first aspect of the invention, the plurality of piezoelectric elements are arranged concentrically and equidistantly.

In the radial array ultrasound endoscopic probe according to the first aspect of the invention, each of the plurality of piezoelectric elements has a width not greater than 40% of its height.

In the radial array ultrasound endoscopic probe according to the first aspect of the invention, the plurality of piezoelectric elements are provided on a first cylindrical segment of the metal cylinder, the circular lattice is provided on a second cylindrical segment of the metal cylinder which is adjacent to the plurality of piezoelectric elements, and the metal cylinder at least has a exposed third cylindrical segment which is adjacent to the second cylindrical segment.

In the radial array ultrasound endoscopic probe according to the first aspect of the invention, the gears of the circular lattice has a same number as the number of the piezoelectric elements, and are arranged equidistantly and concentrically; each of the plurality of coaxial cables is correspondingly placed and fixed in one gear.

In the radial array ultrasound endoscopic probe according to the first aspect of the invention, the inner region of the ceramic or single crystal ring that exceeds the backing material layer is coated with conductive adhesive, such that the inner electrode of the piezoelectric ring is electrically connected with the metal cylinder; and the conductive adhesive is coated with insulating adhesive; the cores and the grounding wires of the coaxial cables are separated to the right and left sides of the circular lattice; the cores of all coaxial cables are connected to the marginal region of the outer electrode of the piezoelectric ring through conductive adhesive; the grounding wires of all coaxial cables are connected to the metal cylinder at the center through conductive adhesive.

In the second aspect of the invention, it is provided a manufacture method for a radial array ultrasound endoscopic probe, which comprises:

S1: the inner and outer surfaces of the piezoelectric ceramic single crystal ring are respectively plated with electrode and polarized;
S2: a backing material is filled to a metal cylinder, such that its diameter is equal to the inner diameter of the piezoelectric ring, and the length of the backing material is less than that of the piezoelectric ring;
S3: the metal cylinder coated with the backing material is adhered to the inner of the piezoelectric ring through insulating adhesive;
S4: the outer electrode of the ring is coated with solvable adhesive at the marginal region thereof for leading;
S5: the outer electrode of the ring is filled with a matching material layer, such that the thickness of the matching material layer amounts to a design value;
S6: the inner electrode of the ring is electrically connected with the metal cylinder at the center through conductive adhesive, and the surface of the conductive adhesive is coated with insulating adhesive for protection;
S7: the solvable adhesive is stripped off to expose the marginal region of the outer electrode of the piezoelectric ring;
S8: the circular plastic ring is cut so as to obtain a circular lattice in a gear shape, wherein the number of teeth of the lattice is equal to the number of the piezoelectric elements of the ultrasonic endoscopic probe, and the diameter of the circular plastic ring is slightly larger than the outer diameter of the piezoelectric ceramic or single crystal ring;
S9: the circular lattice is passed through the metal cylinder so as to abut with the exposed electrode of the piezoelectric ring, and is fixed to the metal cylinder;
S10: the coaxial cables of the same number as the piezoelectric elements of the ultrasound endoscopic probe is deployed within the concave grooves of the circular lattice, the core and the grounding wire of each coaxial cable are stripped off to expose the metal wire part, and are separated to the each side of the circular lattice;
S11: the cores of the coaxial cables are connected to the marginal region of the outer electrode of the piezoelectric ring through conductive adhesive, the grounding wires of the coaxial cables are connected to the metal cylinder through conductive adhesive, and the surface of the conductive adhesive is coated with insulating adhesive for protection;
S12: a plurality of piezoelectric elements arranged in a circle are cut out from the piezoelectric ceramic or single crystal ring which is covered with a matching material layer, wherein the cutting depth is such that the piezoelectric ring is cut through, and the cutting grooves are filled with decoupling material to solidify therein.

In the manufacture method for a radial array ultrasound endoscopic probe according to the second aspect of the invention, in Step S8, the gears of the circular plastic rings that are cut out are arranged concentrically, and equidistantly, and in Step S12, the plurality of piezoelectric elements that are cut out are arranged concentrically and equidistantly.

In the third aspect of the invention, it is provided a fixing and rotating device of the radial array ultrasound endoscopic probe as mentioned above, comprising a fixing support that protrudes out of the platform of the fixing device, and the fixing support is provided on one side thereof with a rotary head for fixing and rotating a workpiece on the other side to rotate within 360 degrees.

In the forth aspect of the invention, it is provided with a cutting method for the radial array ultrasound endoscopic probe as mentioned above, wherein the radial array ultrasound endoscopic probe is fixed by means of the fixing and rotating device according to claim 10, and is placed in a mechanical linear cutting apparatus or a laser cutting apparatus for cutting.

The radial array ultrasound endoscopic probe, the manufacture method thereof and the fixing and rotating device embodying the invention present the following advantages. Instead of cutting out all piezoelectric elements first and then arranging them in a circle as commonly used in the prior art, the plurality of piezoelectric elements according to the invention are directly cut out from the piezoelectric ceramic or single crystal ring. In this way, problems that may otherwise arise from forcedly curling multiple layers of materials of a certain thickness into a circular tube, such as non-concentricity of elements, misalignment of elements at interface, break-off of piezoelectric ceramic or single crystal, disengagement of the acoustic matching layer and the backing layer, and breaking of connection of welding points etc., are avoided. Moreover, instead of employing a flexible circuit broad for connecting signal lines as commonly used in the prior art, the circular lattice in a gear shape is utilized to deploy the signal lines of coaxial cables. In this way, all signal lines are concentrically and equidistantly aligned with the respective elements. Furthermore, spot welding is dispensed with for each element and signal line. That is, all cores are coated with conductive adhesive, connected to the piezoelectric elements, and respective signal lines are electrically disconnected in the following elements cutting process.

### BRIEF DESCRIPTION OF THE DRAWINGS

Hereinafter the invention will be further described with reference to the embodiments and the companying figures, wherein

FIG. 1 is a perspective structural view showing the radial array ultrasound endoscopic probe according to the preferable embodiment of the invention;

FIG. 2 is a longitudinal cross sectional view showing the radial array ultrasound endoscopic probe according to the preferable embodiment of the invention;

FIG. 3 is a perspective view showing the circular lattice of the radial array ultrasound endoscopic probe according to the preferable embodiment of the invention;

FIG. 4 is the flow chart of the manufacture method for the radial array ultrasound endoscopic probe according to the preferable embodiment of the invention [401]: design and manufacture piezoelectric ceramic or single crystal ring; 402: the inner and outer surfaces of the piezoelectric ceramic or single crystal ring are respectively plated with electrode and polarized; 403: a backing material is filled to a metal cylinder in a special mold, and the solidified cylinder is machined such that its diameter is equal to the inner diameter of the piezoelectric ceramic or single crystal ring; 404: the backing cylinder is adhered to the inner of the piezoelectric ceramic or single crystal ring through epoxy resin; 405: the outer electrode of the piezoelectric ceramic or single crystal ring is coated with solvable adhesive at the marginal region thereof for leading; 406: the outer electrode of the piezoelectric ceramic or single crystal ring is filled with a matching material layer in a special mold, and the solidified cylinder is machined such that the thickness of the matching material layer amounts to a design value; 407: the inner electrode of the piezoelectric ceramic or single crystal ring is electrically connected with the metal cylinder through conductive adhesive; 408: the solvable adhesive is stripped off to expose the electrode at the outer marginal region of the piezoelectric ceramic or single crystal ring; 409: on a special fixing and rotating device, the circular plastic ring is cut using liner cutting machine so as to obtain a circular lattice in a gear shape, wherein the number of teeth of the circular lattice is equal to the number of the piezoelectric elements of the ultrasonic endoscopic probe; 410: the circular lattice in a gear shape is closely fixed on the side of the piezoelectric ceramic or single crystal ring on which the electrode is expose; 411: the coaxial cables of the same number as the piezoelectric elements of the ultrasound endoscopic probe is deployed within the concave grooves of the circular lattice; 412: the cores of the coaxial cable are connected to the electrode at the outer marginal region of the piezoelectric ceramic or single crystal ring through conductive adhesive, and the grounding wires of the coaxial cables are connected to the metal cylinder at the center through conductive adhesive; 413: on a special fixing and rotating device, the radial arrays are cut out using linear cutting machine, wherein the cutting grooves are filled with decoupling material to solidify therein, and excessive portion of the metal cylinder is cut off, and the radial array is placed on the leading end of an endoscopy.];

FIG. 5a is a perspective view showing the fixing and rotating device for the radial array ultrasound endoscopic probe according to the preferable embodiment of the invention;

FIG. 5b is a side view showing the fixing and rotating device for the radial array ultrasound endoscopic probe according to the preferable embodiment of the invention;

FIG. 6 shows cutting action conducted by the radial array ultrasound endoscopic probe according to the preferable embodiment of the invention.

### DETAILED DESCRIPTION OF THE INVENTION

The invention will be explained in detail with reference to the accompanying figures and embodiments hereinafter to further clarify the purposes, the technical solutions and advantages of the invention.

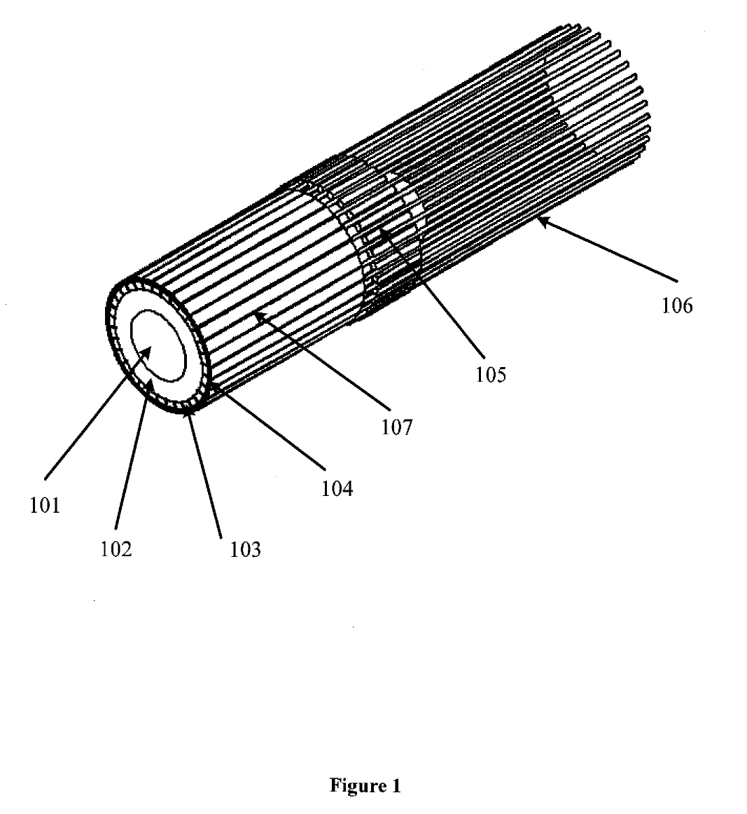

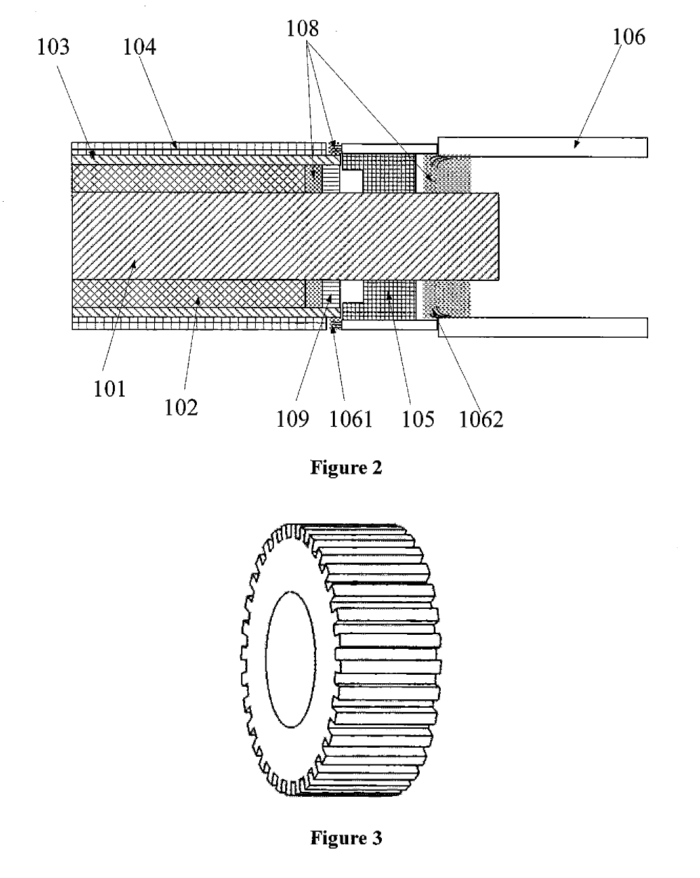

As shown in FIGS. 1 and 2 which illustrate the structure of the radial array ultrasound endoscopic probe according to the preferable embodiment of the invention in a perspective view and a longitudinal cross sectional view, respectively, the radial array ultrasound endoscopic probe according to the preferable embodiment includes a metal cylinder 101, a backing material layer 102, piezoelectric elements 103, a matching material layer 104, a circular lattice 105, coaxial cables 106 and decoupling material 107.

The metal cylinder 101 is located at the center and functions as a support, and connects the electrodes of the piezoelectric elements 103 and the grounding wires of all coaxial cables 106. It is preferred, but not necessary, that the metal cylinder 101 was made of bronze due to its good machinability and conductivity.

A plurality of piezoelectric elements 103 are arranged concentrically and equidistantly around the metal cylinder 101. Various piezoelectric ceramic and piezoelectric single crystal materials may be utilized to prepare the piezoelectric elements. Furthermore, the number of the piezoelectric elements may be 32, 64, 128 or even more, but is not limited thereto in the present invention.

The backing material 102 is provided between the piezoelectric elements 103 and the metal cylinder 101. It functions to absorb ultrasound waves transmitted backwards from the piezoelectric elements 103 so as to improve the imaging resolution of the probe.

The matching material layer 104 covers around the periphery of the piezoelectric elements 103. The matching material layer 104 may consists of a single layer, two layers or a plurality of layers. The thickness and the acoustic parameters of the matching materials 104 may be designed according to the working frequency, electrical and acoustic parameters of the piezoelectric elements 103.

Each piezoelectric element 103 is connected to a very thin coaxial cable 106 so as to transmit excitation voltage signals and receive echo wave voltage signals. The diameter of coaxial cable 106 is small than or equal to the width of the piezoelectric elements 103.

The circular lattice 105 is in the shape of a gear so as to arrange the coaxial cables 106 and separate the cores and grounding wires thereof. It also conducts locating function during cutting the piezoelectric elements. As shown in FIG. 3 which is a perspective view showing the circular lattice in the radial array ultrasound endoscopic probe according to the preferable embodiment of the invention, the number of gears (teeth) of the circular lattice 105 is equal to that of the piezoelectric elements 103, and all gears are preferably concentric and equally distanced.

Decoupling material 107 is provided between each piezoelectric element 103 so as to decrease the crosstalk effect among the piezoelectric elements 103.

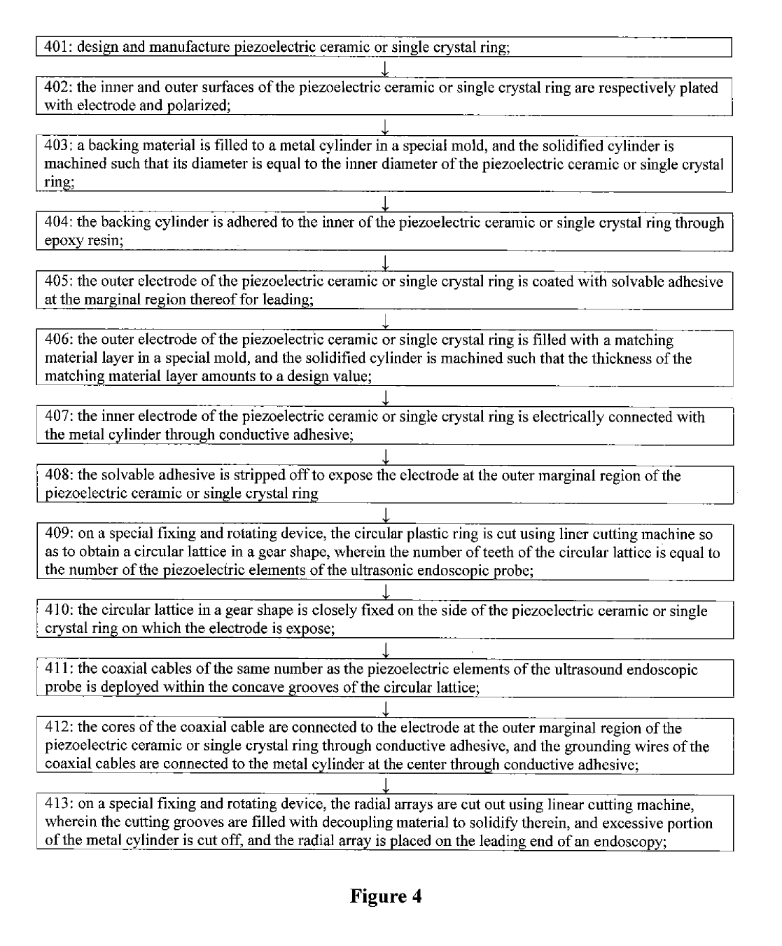

Now turn to FIG. 4 showing flow chart of the manufacture method for the radial array ultrasound endoscopic probe according to the preferable embodiment of the invention. Hereinafter, the manufacture method for the radial array ultrasound endoscopic probe as proposed in the embodiment will be described with reference to the inner structure as shown in FIG. 2.

First, in Step 401, a ring 103 made of piezoelectric ceramic or single crystal materials is prepared. Due to the space confinement from the endoscopy specific for a certain body-cavity, the diameter and the height of the ring 103 could not be too large. The wall thickness of the ring 103 should be properly chosen according to the working frequency of the probe actual in use and the frequency of the piezoelectric materials in such a way that the higher the frequencies, the smaller the wall thickness. Generally speaking, the ring 103 is machined from bulk piezoelectric ceramic or single crystal, since the thus obtained ring may have good concentricity.

In Step 402, electrodes are prepared on the inner and outer surfaces of the piezoelectric ceramic or single crystal ring 103 through vacuum sputtering coating, chemical plating or electroplating. The ring is subject to polarization so as to have piezoelectric performance.

In Step 403, a metal cylinder 101 is further prepared, the diameter of which is slightly larger than the inner diameter of the plastic pipe as shown in FIG. 3 and is less than 5 μm. The left side of the metal cylinder 101, i.e., the first segment, is prepared with a loop of backing material 102, the length of which is less than that of the ring 103, and the outer diameter of which is equal to the inner diameter of the ring 103. The backing material 102 may be prepared as follows. After the metal cylinder 101 is located in a special mold, the liquid dispersed with epoxy resin, sound-absorbing rubber, coarse granular powder and heavy granular powder is filled therein, and it should be ensured that the height of the backing material that have been filled should be less than that of the ring 103. It should be appreciated that the invention has no limit on the formula of the backing material. Then the backing material is drawn out of the mold after curing. Finally, the backing material is machined to strip off the unwanted circumferential part such that its outer diameter is equal to the inner diameter of the ring 103.

In Step 404, the backing material is uniformly coated with adhesive (such as epoxy resin) having good fluidity and high cohesion strength, and then the ring 103 is sleeved over the backing material 102. The backing material and the ring will combine together after solidification.

In Step 405, a small length (the location to be coated with conductive adhesive 1061) of electrode located peripherally on the right side of the ring 103 is coated with a narrow band of adhesive. The adhesive may be alkyd, which may solidify within atmosphere environment at room temperature and may be removed by means of solvents such as acetone. The purpose of coating a band of adhesive is to protect the coated electrode from being screened by the material of the matching layer, and to allow the peripheral electrode of the ring 103 to connect to the coaxial cable signal lines.

In Step 406, the ring 103 adhered with the backing material is positioned within a special mold, and the material of matching layer that has been properly formulated is filled therein. The matching layer and the ring adhered with the backing material are drawn out together after solidification. It should be ensured that the height of the matching material layer 104 filled therein does not exceed the band of adhesive (alkyd) that have already been coated. The acoustic impedance of the matching material layer 104 is chosen as a function of those of the specific piezoelectric ceramic or single crystal and body tissues, and its thickness depends on the working frequency of the probe. The solidified cylinder is machined to the extent that it assumes a proper diameter such that the thickness of matching material layer is equal to the calculated value. There may be designed and prepared a single layer, two layers, or even more layers of matching material, and the invention imposes no limit thereto.

In Step 407, the inner region of the ring 103 that protrudes beyond the backing material 102 is coated with conductive adhesive 108 such that the inner electrode of the ring 103 electrically connects with the metal cylinder 101 located centrally. And the adhesive 108 is coated with an adhesive 109 (such as epoxy resin, etc.) to avoid electrical connection between the inner and outer electrodes.

In Step 408, the band of adhesive (alkyd) that has been coated and solidified is removed through acetone such that the electrode coated with the adhesive is exposed.

In Step 409, a circular plastic ring is cut out on a special fixing and rotating device using a mechanical linear cutting apparatus or a laser cutting apparatus so as to obtain a circular lattice in the shape of a gear structure with the number of teeth being equal to the number of the piezoelectric elements of the ultrasound endoscopic probe.

In Step 410, the gear-shaped circular lattice 105 prepared as described in FIG. 3 is penetrated through the metal cylinder 101 to tightly abut against the ring 103, and is fixed with adhesive (such as epoxy resin, etc.).

In Step 411, the coaxial cables 106, the number of which is equal to the number of the piezoelectric elements of the probe, are located in the circular lattice 105 in order, and are fixed with adhesive.

In Step 412, the cores 1061 and grounding wires 1062 of the coaxial cables 106 are each stripped off for a small fraction of metal wires to be exposed, and are separated by the circular lattice 105 at both sides. The exposed part of the cores is such that it overlaps over the electrode portion of the ring 103 that is exposed outside; and conductive adhesive 108 is employed to adhere the exposed part of the cores with the said electrode portion of the ring 103 that is exposed outside; then an adhesive layer (such as epoxy resin, etc.) is coated thereon for protection. The grounding wires 1062 of the coaxial cables 106 are adhered to the metal cylinder 101 through conductive adhesive, and are electrically connected with the inner electrodes of the ring 103 through the metal cylinder 101. Likewise, an adhesive layer is coated on the conductive adhesive for protection.

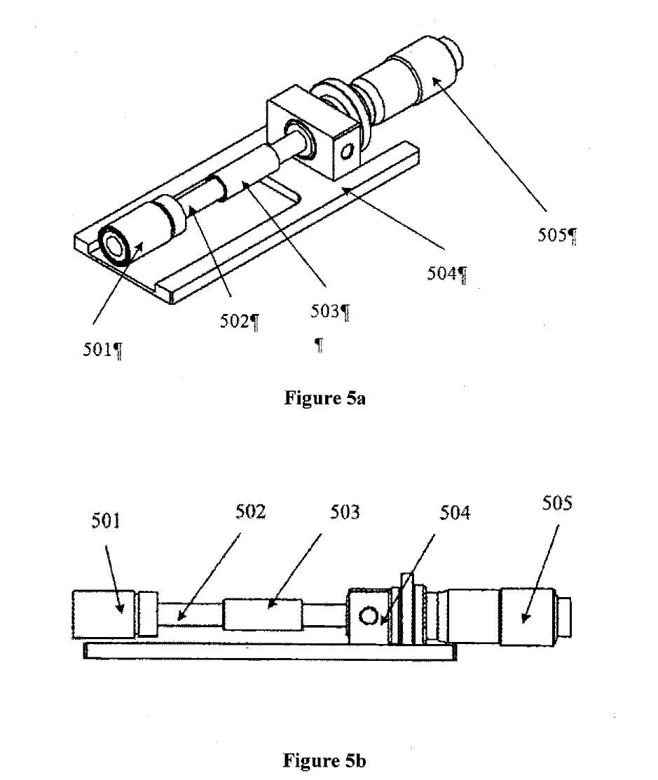

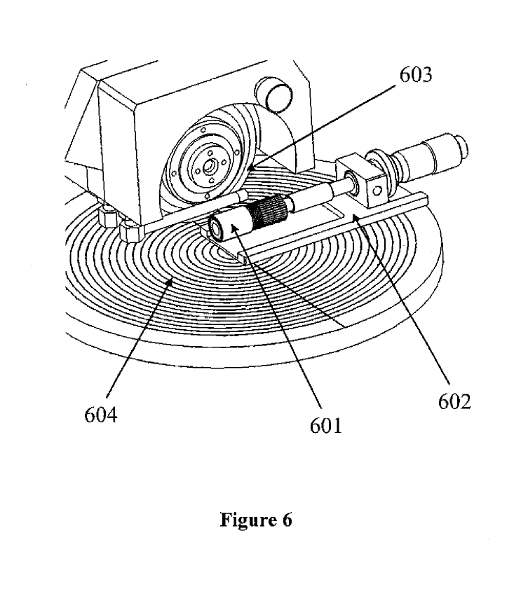

In Step 413, the thus prepared workpiece is installed to the fixing and rotating device as shown in FIGS. 5a and 5b, and is fixed to the cutting platform of a cutting machine as shown in FIG. 6 through vacuum sorption. Even though a mechanical linear cutting apparatus is shown in the figures, the invention is not limited thereto by any means, e.g., a laser cutting apparatus may be employed. One wire cutting is performed per rotation of 360°/N, that is, cutting is performed N times per cycle. The cutting depth is such that the ring 103 is just cut through to the backing material 102, and thus N piezoelectric elements are formed. Each piezoelectric element has a width at least less than 40% of the wall thickness of the ring 103. In this way it is ensured that the vibration mode thereof possesses a relatively high mechanical-electrical transfer coefficient. Gaps between each piezoelectric element are filled with decoupling material which will solidify. The excessive portion of the third segment of the metal cylinder 101 to the right side of the ring 103 is cut off such that it is obtained a radial array ultrasound endoscopic probe as shown in FIG. 5, which can be installed on the leading end of an endoscopy with all coaxial cables 106 being connected to the host computer through the conduit of the endoscopy.

Turn to FIGS. 5a and 5b which show the perspective view and side view of the fixing and rotating device of the radial array ultrasound endoscopic probe according to the preferable embodiment of the invention, respectively. The fixing and rotating device is used in conjunction with a mechanical linear cutting apparatus or a laser cutting apparatus to perform a rotary cutting action with high precision. The workpiece to be fixed and rotated is designated by 501, which, in the invention, maybe the main body of a radial array probe before being cut into an array form, or a plastic circular ring before being cut into a gear shape. The metal cylinder is designated by 502, which may be a supporting metal cylinder at the center of the radial array probe, or a single metal cylinder functioning to fix the circular plastic ring. The rotary head is designated by 505, which is provided with a scale such that the rotation within a range of 360 degrees can be precisely controlled. The rotary head is fixed to the fixing base 504, with the portion of the metal cylinder protruding on the left side thereof passing through the circular hole at the center of the fixing base 504 and connecting to the circular plastic pipe 503. The inner diameter (less than 5 microns) of the circular plastic pipe 503 is slightly less than the outer diameters of the metal cylinders at the left and right sides, such that a naturally tight state may result when putting the metal cylinder into the circular plastic pipe. In this way, the workpiece on the left side can be driven to precisely rotate through rotating the rotary head on the right side. It should be noted that, to achieve a proper cutting precision, each component should be concentric. The bottom of the fixing base is a smooth surface, which may be absorbed to the cutting platform of the mechanical linear cutting apparatus or the laser cutting apparatus by means of vacuum.

Now turn to FIG. 6 which is a schematic view showing a cutting action made to the radial array ultrasound endoscopic probe according to the preferable embodiment of the invention. Even though the example of employing a mechanical linear cutting apparatus is shown, the invention is not limited thereto in that a laser cutting apparatus may be equivalently employed. As shown in FIG. 6, the main body of the radial array ultrasound endoscopic probe before performing linear cutting is designated by 601, the fixing and rotating device is designated by 602, the edge of the linear cutting apparatus is designated by 603, and the cutting platform of the linear cutting apparatus is designated by 604. The special fixing and rotating device fabricated by the present invention can incorporate with the cutting apparatus so as to cut out a proper circular lattice and piezoelectric elements. For example, a circular lattice in a gear shape can be produced using the cutting apparatus in conjunction with the fixing and rotating device designed according to FIG. 5. For instance, a circular plastic ring can be machined, with its outer diameter being slightly greater than the outer diameter of the piezoelectric ring and its inner diameter being equal to the outer diameter of the metal cylinder. Plastic materials which are easy to cut can be selected for the ring. The circular plastic ring is sleeved over the metal cylinder and fixed to the fixing and rotating device 602. The bottom of the base of the fixing and rotating device is absorbed to the cutting platform 604 of the cutting apparatus via vacuum. An angle of 360°/N is assigned to each of arrays with the N (N may assume 32, 64, 128 or even more, without limitation from the invention, per se) elements of the radial array ultrasound probe being one 360 degree circle. One linear cutting is performed for each 360°/N rotation. The cutting parameters should be properly chosen such that the width of cutting grooves is slightly greater than the diameter of inner cores of the coaxial cable (thereafter, the coaxial cable signal lines can be deployed within the cutting grooves), and the depth of cutting grooves is such that the distance from the bottom of the cutting grooves to the center of the ring is slightly less than outer diameter of the piezoelectric ceramic or single crystal tube. The structure as shown in FIG. 3 will result after completion of N cutting actions of one cycle.

The radial array ultrasound endoscopic probe provided by the invention, used in conjunction with an endoscope, can electronically scan the surrounding tissues and organs panoramically within human alimentary canals so as to pick up a radial ultrasound image of the surrounding tissues and organs. The invention possesses the following advantages.

A Simple Manufacture Process

Instead of cutting out all piezoelectric elements first and then arranging them in a circle as commonly used in the prior art, the plurality of piezoelectric elements according to the invention are directly cut out from the piezoelectric ceramic or single crystal ring. In this way, problems that may otherwise arise from forcedly curling multiple layers of materials of a certain thickness (at least including an acoustic matching layer, a piezoelectric layer, and a backing layer) into a circular tube, such as non-concentricity of arrays, misalignment of arrays at interface, break-off of piezoelectric ceramic or single crystal, disengagement of the acoustic matching layer and the backing layer, and breaking of connection of welding points etc., are avoided.

Instead of employing a flexible circuit broad for connecting signal lines as commonly used in the prior art, the circular lattice in a gear shape is utilized to deploy the signal lines of coaxial cables. In this way, all signal lines are concentrically and equidistantly aligned with the elements equal-distantly. Furthermore, spot welding is dispensed with for each element and signal line. That is, all cores are coated with conductive adhesive, connected to the piezoelectric elements, and electrically disconnected in the following array cutting process.

A Reliable Manufacture Process

The desired thickness is achieved through machining after filling the special mold with the backing material and the matching material. Thus it is ensured that each layer of material has a uniform thickness, can be firmly engaged with each other, and the surface of the probe is a perfect smooth cylinder.

A structure of a circular plastic pipe capable of naturally tightening is employed to connect the uncut radial array probe without adhesive or connecting mechanism (such as threads) with the rotary head. In this way the uncut probe and the rotary head may rotate in a concentric state so as to ensure the precision of rotary cutting.

The special fixing and rotating device is used with a cutting apparatus, such that the piezoelectric ring, the matching layer and the backing layer fixed on the piezoelectric ring can be controlled to rotate precisely. Equally distanced elements with equal width may result from cutting.

The invention is described with reference to the specific embodiments. Nevertheless, the skilled in the art will appreciate that there may exit various changes and equivalent substitutions without departing the scope of the invention. In addition, various modifications can be made to the invention for adapting to specific application fields or materials, without departing from the protection scope of the invention. Therefore, the invention is not limited to specific embodiments as disclosed herein, and to the contrary, the invention includes all embodiments falling within the protection scope as defined by the claims

What is claimed is:

1. A radial array ultrasound endoscopic probe, characterized in that said probe comprises:
a metal cylinder located at the center;
a plurality of piezoelectric elements which are cut from piezoelectric ceramic or single crystal rings and are arranged around the metal cylinder, said piezoelectric elements and said metal cylinder being provided with a backing material layer for absorbing sound therebetween, said piezoelectric elements being covered with a matching material layer on the outer side thereof, and said piezoelectric elements are filled with decoupling material therebetween;
a plurality of coaxial cables correspondingly connected with the plurality of piezoelectric elements, the grounding wire of each coaxial cable being connected to the metal cylinder; and
a circular lattice in a gear shape which sleeves over said metal cylinder and functions to arrange and separate the coaxial cables.

2. The radial array ultrasound endoscopic probe according to claim 1, characterized in that, the plurality of piezoelectric elements are arranged concentrically and equidistantly.

3. The radial array ultrasound endoscopic probe according to claim 1, characterized in that, each of the plurality of piezoelectric elements has a width not greater than 40% of its height.

4. The radial array ultrasound endoscopic probe according to any one of claims 1 to 3, characterized in that, the plurality of piezoelectric elements are provided on a first cylindrical segment of the metal cylinder, the circular lattice is provided on a second cylindrical segment of the metal cylinder which is adjacent to the plurality of piezoelectric elements, and the metal cylinder at least has a exposed third cylindrical segment which is adjacent to the second cylindrical segment.

5. The radial array ultrasound endoscopic probe according to claim 4, characterized in that, the gears of the circular lattice has a same number as the number of the piezoelectric elements, and are arranged equidistantly and concentrically; each of the plurality of coaxial cables is correspondingly placed and fixed in one gear.

6. The radial array ultrasound endoscopic probe according to claim 5, characterized in that, the inner region of the piezoelectric ceramic or single crystal ring that exceeds the backing material layer is coated with conductive adhesive, such that the inner electrode of the piezoelectric ring is electrically connected with the metal cylinder; and the conductive adhesive is coated with insulating adhesive.

7. The radial array ultrasound endoscopic probe according to claim 5, characterized in that, the cores and the grounding wires of the coaxial cables are separated to the right and left sides of the circular lattice; the cores of all coaxial cables are connected to the marginal region of the outer electrode of the piezoelectric ring through conductive adhesive; the grounding wires of all coaxial cables are connected to the metal cylinder at the center through conductive adhesive.

8. A manufacture method for the radial array ultrasound endoscopic probe according to any one of claims 1-7, characterized in that said method comprises the following steps,
S1: the inner and outer surfaces of the piezoelectric ceramic ring or piezoelectric single crystal ring are respectively plated with electrode and polarized;
S2: a backing material is filled to a metal cylinder, such that its diameter is equal to the inner diameter of the piezoelectric ring, and the length of the backing material is less than that of the piezoelectric ring;
S3: the metal cylinder coated with the backing material is adhered to the inner of the piezoelectric ring through insulating adhesive;
S4: the outer electrode of the piezoelectric ring is coated with solvable adhesive at the marginal region thereof for leading;
S5: the outer electrode of the piezoelectric ring is filled with a matching material layer, such that the thickness of the matching material layer amounts to a design value;
S6: the inner electrode of the piezoelectric ring is electrically connected with the metal cylinder at the center through conductive adhesive, and the surface of the conductive adhesive is coated with insulating adhesive for protection;
S7: the solvable adhesive is stripped off to expose the marginal region of the outer electrode of the piezoelectric ring;
S8: the circular plastic ring is cut so as to obtain a circular lattice in a gear shape, wherein the number of teeth of the circular lattice is equal to the number of the piezoelectric elements of the ultrasonic endoscopic probe, and the diameter of the circular plastic ring is slightly larger than the outer diameter of the piezoelectric ceramic single crystal ring;
S9: the circular lattice is passed through the metal cylinder so as to abut with the exposed electrode of the piezoelectric ring, and is fixed to the metal cylinder;
S10: the coaxial cables of the same number as the piezoelectric elements of the ultrasound endoscopic probe is deployed within the concave grooves of the circular lattice, the core and the grounding wire of each coaxial cable are stripped off to expose the metal wire part, and are separated to the each side of the circular lattice;
S11: the cores of the signal lines of the coaxial cables are connected to the marginal region of the outer electrode of the piezoelectric ceramic or single crystal ring through conductive adhesive, the grounding wires of the coaxial cables are connected to the metal cylinder through conductive adhesive, and the surface of the conductive adhesive is coated with insulating adhesive for protection;
S12: a plurality of piezoelectric elements arranged in a circle are cut out from the piezoelectric ceramic or single crystal ring which is covered with a matching material layer, wherein the cutting depth is such that the piezoelectric ring is cut through, and the cutting grooves are filled with decoupling material to solidify therein.

9. The manufacture method for the radial array ultrasound endoscopic probe according to claim 8, characterized in that, in Step S8, the gears of the circular plastic rings that are cut out are arranged concentrically and equidistantly, and in Step S12, the plurality of piezoelectric elements that are cut out are arranged concentrically and equidistantly.

10. A fixing and rotating device of the radial array ultrasound endoscopic probe according to any one of claims 1-7, characterized in that it comprises a fixing support that protrudes out of the platform of the fixing device, and the fixing support is provided on one side thereof with a rotary head for fixing and rotating the workpiece on the other side to rotate within 360 degrees.

11. A cutting method for the radial array ultrasound endoscopic probe according to any one of claims 1-7, characterized in that, the radial array ultrasound endoscopic probe is fixed by means of the fixing and rotating device according to claim 10, and is placed in a mechanical linear cutting apparatus or a laser cutting apparatus for cutting.
